(19) United States
(12) Patent Application Publication
(10) Pub. No.: US 2022/0058972 A1
(43) Pub. Date: Feb. 24, 2022

(54) System and Method for Enhanced Retention of Procedure-Related Information

(71) Applicants: Antonakis Maritis, Santa Monica, CA (US); Antonio Wilson, Santa Monica, CA (US)

(72) Inventors: Antonakis Maritis, Santa Monica, CA (US); Antonio Wilson, Santa Monica, CA (US)

(21) Appl. No.: 16/996,631

(22) Filed: Aug. 18, 2020

(51) Int. Cl. G09B 5/06; G06T 13/40; G09B 19/00; G16H 80/00

(52) U.S. Cl. CPC G09B 5/065; G06T 13/40; G06T 2210/41; G16H 80/00; G09B 19/00

(57) ABSTRACT

A system and method for enhanced retention of procedure-related information is provided. In some embodiments, the system and method may comprise a computing device associated with a medical professional and a computing device associated with a patient. The medical professional computing device may store one or more medical procedure videos, receive identity information associated with the patient, assign and transmit the one or more medical procedure videos to the patient, and detect and display a patient completion, wherein the patient completion comprises the patient viewing the one or more medical procedure videos. The patient computing device may receive and transmit the identity information associated with the patient, receive and display the one or more medical procedure videos, and notify and transmit the patient completion.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to lighting systems and, more particularly, to a linear lighting system and apparatus wherein one or more lighting assemblies are contiguously joined together using a draw latch assembly so as to reduce installation cost and time.

### BACKGROUND

The entire healthcare system relies on the assumption that patients can understand and retain complex written and spoken health information. Health literacy is defined as the degree to which individuals have the capacity to obtain, process, and understand basic health information and services needed to make appropriate medical decisions. Limited health literacy can affect health status, health outcomes, health care use, and health costs. Much of the time, even well-educated individuals experience trouble understanding medication instructions, informed consent, procedure-related information, discharge instructions, and health education materials. This, in turn, may lead to a lack of proper health self-management and poor health decision-making. To make matters worse, individuals often experience even greater difficulty understanding medical procedures, as well as, preoperative and postoperative care and instructions. Thus, there remains a need for a system and method which can enhance retention of health and procedure-related information.

In a moving picture or video, a frame is one of many still images which compose the complete moving picture or video. While motion can be detected at far lesser frame rates, most videos play at twenty-four frames per second. In other scenarios, such as virtual reality, gaming, sports, and reality programming, videos may play at an even faster pace of up to sixty frames per second. For entertainment purposes, these high frame rates remain adequate for a proper understanding of a plot and on-screen events. However, if individuals desire actual learning and retention of information presented on-screen, a lower frame rate may be desirable.

It is known that learning is multi-sensory and that combining both visual and auditory information can increase retention of information. Indeed, animated videos which stimulate both auditory and visual senses may provide the ideal medium for providing information. Further, videos utilizing animation, text, voice, and music in specific configurations maximize retention of information. As a result of the foregoing, there remains a need for videos which combine multiple senses at a slower speed to enhance retention of procedure-related information.

### SUMMARY

The present disclosure is directed to a system and method for enhanced retention of procedure-related information, which provides for at least two computing devices capable of transmitting one or more medical procedure videos to a patient so as to increase the patient's retention of preoperative and postoperative information. The system and method may stimulate both auditory and visual senses, particularly utilizing animation, text, voice, and music in combination to maximize retention. Indeed, the system and method may increase retention of information to 87%. Moreover, retention may be 580% greater than when utilizing text alone and 30% more effective than when utilizing animation, voice, and/or music alone.

In many embodiments, and for purposes of brevity, the system and method for enhanced retention of procedure-related information wherein the one or more medical procedure videos comprise one or more medical procedures, such as a surgical procedure, are shown and described. However, it will be understood that the one or more medical procedure videos may include virtually any type of medical procedure, therapy, or treatment. Moreover, in certain further embodiments, this system and method may apply to other types of information, including non-medical information, as well. For purposes of brevity, the aforementioned information provided via the videos will hereafter be referred to as the “one or more medical procedure videos.”

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, a system for enhanced retention of procedure-related information is provided that may utilize a computing device associated with a medical professional and a computing device associated with a patient. The computing device associated with the medical professional (variously referred to as “medical professional computing device” or “professional computing device”) may be operative to store the one or more medical procedure videos. The one or more medical procedure videos may be generally categorized as preoperative videos, operative videos, and postoperative videos. The one or more medical procedure videos may be further subcategorized as home preparation videos, risk videos, and hospital expectations videos. Also, the one or more medical procedure videos may comprise visual data and audio data.

In some embodiments, the visual data may comprise at least one video screen and the at least one video screen may have fifteen frames. The visual data may further feature one or more of image content, animation content, and text content. The animation content may display at between 5 to 15 frames per second. In certain embodiments, the animation content may display at 8 frames per second. In certain embodiments, the image content, the animation content, or both the image content and the animation content may comprise at least one character. According to some embodiments, the at least one character may resemble a human or an animal. In exemplary embodiments, the at least one character may have a face. In other embodiments, the image content and/or the animation content may comprise medical equipment, tools, instruments, or other items related to a medical procedure.

The text content may comprise one or more words associated with the one or more medical procedures. In some embodiments, the text content may comprise key or highlight words associated with the one or more medical procedures and also, the audio data. The text content may also comprise a full sentence corresponding to the one or more medical procedures and therefore, the audio data. In embodiments where the at least one video screen may comprise three video screens, the text content may comprise up to 24 words across the three screens. In such embodiments, the up to 24 words may comprise the full sentence.

The audio data may comprise speech content and non-speech content. In certain embodiments, the speech content may comprise at least one speaker and the at least one speaker may further comprise a female voice. In further embodiments, the female voice may be a British female voice. Of course, in other embodiments, the at least one speaker may comprise a male voice or a gender-neutral voice as well. Further, the at least one speaker may comprise the female voice, the male voice, or the general-neutral voice as an American voice. In other embodiments, the at least one speaker may comprise the female voice, the male voice, or the general neutral voice as other types of voices based on a language and a dialect of the patient. The non-speech content may comprise instrumental music or computer music. Moreover, the non-speech content may be non-dynamic in that the non-speech content may comprise little or no variation in volume between notes or phrases. In certain embodiments, the non-speech content may not overpower the speech content.

The medical professional computing device may be further operative to receive identity information associated with the patient. The identity information may comprise the one or more medical procedures recommended or prescribed to the patient by the medical professional. The identity information may also comprise personal information about the patient, such as name, medical history, clinical data, diagnoses, medications, treatment plans, and laboratory and test results. The professional computing device may also be operative to assign and transmit the one or more medical procedure videos to the patient. In so doing, the professional computing device may transmit the one or more medical procedure videos to the patient based on the one or more medical procedures recommended or prescribed to the patient by the medical professional.

In some embodiments, the medical professional computing device may be operative to detect and display a patient completion. In such embodiments, the patient completion may comprise the patient viewing the one or more medical procedure videos. Moreover, the patient completion may comprise the patient viewing one or more of the one or more medical procedure videos. In this manner, the patient completion may be defined by the medical professional.

According to certain embodiments, the computing device associated with the patient (also referred to as “patient computing device”) may be operative to transmit the identity information associated with the patient. The patient computing device may also be operative to receive and display the one or more medical procedure videos. In addition, the patient computing device may be further operative to notify and transmit the patient completion.

In further embodiments, the method may comprise the steps of providing a computing device associated with a medical professional and a computing device associated with a patient; storing, at the computing device associated with the medical professional, one or more medical procedure videos; receiving, at the computing device associated with the medical professional, identity information associated with the patient; assigning and transmitting, at the computing device associated with the medical professional, the one or more medical procedure videos to the patient; detecting and displaying, at the computing device associated with the medical professional, a patient completion, wherein the patient completion comprises the patient viewing the one or more medical procedure videos; transmitting, at the computing device associated with the patient, the identity information associated with the patient; receiving and displaying, at the computing device associated with the patient, the one or more medical procedure videos; and notifying and transmitting, at the computing device associated with the patient, the patient completion.

In some embodiments, receiving, at the medical professional computing device, identity information associated with the patient may further comprise receiving the one or more medical procedures recommended or prescribed by the medical professional to the patient. In other embodiments, receiving the identity information may also comprise receiving personal information about the patient, including name, medical history, diagnoses, laboratory and test results, and treatment plans. Moreover, assigning and transmitting, at the medical professional computing device, the one or more medical procedure videos to the patient may further comprise determining which of the one or more medical procedure videos to assign to the patient. Determining which of the one or more medical procedure videos to assign may depend on the identity information, such as the diagnoses or the treatment plan set forth by the medical professional.

Detecting, at the professional computing device, the patient completion may comprise the patient viewing the one or more medical procedure videos. In certain embodiments, the patient completion may comprise the patient viewing all of the one or more medical procedure videos. In alternate embodiments, the patient completion may comprise the patient viewing one or more of the one or more medical procedure videos. In addition, the patient completion may be determined by the medical professional.

Transmitting, at the patient computing device, the identity information associated with the patient may further comprise receiving, at the patient computing device, an invitation. The invitation may comprise a link or request to access the one or more medical procedure videos and the invitation may further require inputting of a passcode, password, or other information by the patient. The invitation may ensure the correct patient views the correct medical procedure videos.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 shows an embodiment of the system for enhanced retention of procedure-related information.

FIG. 2 shows an embodiment of the method for enhanced retention of procedure-related information.

FIG. 3 shows an embodiment of the method for enhanced retention of procedure-related information.

FIG. 4 shows an embodiment of the method for enhanced retention of procedure-related information.

FIG. 5 shows an embodiment of the system for enhanced retention of procedure-related information.

FIG. 6 shows an embodiment of the system for enhanced retention of procedure-related information.

One embodiment of the invention is implemented as a program product for use with a computer system. The program(s) of the program product defines functions of the embodiments (including the methods described herein) and can be contained on a variety of computer-readable storage media. Illustrative computer-readable storage media include, but are not limited to: (i) non-writable storage media (e.g., read-only memory devices within a computer such as CD-ROM disks readable by a CD-ROM drive) on which information is permanently stored; (ii) writable storage media (e.g., floppy disks within a diskette drive or hard-disk drive) on which alterable information is stored. Such computer-readable storage media, when carrying computer-readable instructions that direct the functions of the present invention, are embodiments of the present invention. Other media include communications media through which information is conveyed to a computer, such as through a computer or telephone network, including wireless communications networks. The latter embodiment specifically includes transmitting information to/from the Internet and other networks. Such communications media, when carrying computer-readable instructions that direct the functions of the present invention, are embodiments of the present invention. Broadly, computer-readable storage media and communications media may be referred to herein as computer-readable media.

In general, the routines executed to implement the embodiments of the invention, may be part of an operating system or a specific application, component, program, module, object, or sequence of instructions. The computer program of the present invention typically is comprised of a multitude of instructions that will be translated by the native computer into a machine-readable format and hence executable instructions. Also, programs are comprised of variables and data structures that either reside locally to the program or are found in memory or on storage devices. In addition, various programs described hereinafter may be identified based upon the application for which they are implemented in a specific embodiment of the invention. However, it should be appreciated that any particular program nomenclature that follows is used merely for convenience, and thus the invention should not be limited to use solely in any specific application identified and/or implied by such nomenclature.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

A system and method for enhanced retention of procedure-related information is provided that, in some embodiments, may allow a medical professional to provide procedure-related information, and particularly, information related to one or more medical procedures, to a patient, thereby maximizing the extent to which the patient retains the information. Moreover, the present system and method optimizes the patient's physical, mental and emotional outcome following the one or more medical procedures because the patient is able to adequately prepare in advance and retain accurate preoperative, operative, and postoperative expectations. As discussed throughout this disclosure, the system and method may involve the medical professional and the patient. However, this system and method may apply to other scenarios involving the desirability of information retention, including those unrelated to medical procedures or settings.

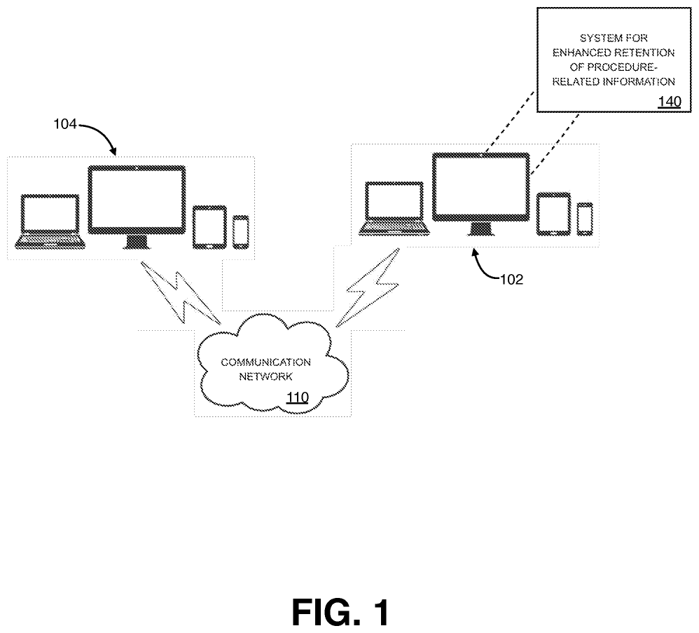

FIG. 1 is illustrative of a networked environment 100 in which an exemplary embodiment of the system for enhanced retention of procedure-related information is implemented. As shown in FIG. 1, system 140 may comprise a computing device associated with the medical professional 102 (alternatively, “medical professional computing device” or “professional computing device”) and a computing device associated with the patient 104 (alternatively, “patient computing device”). By way of example, and not limitation, the medical professional computing device 102 and the patient computing device 104 are shown communicatively coupled via a communication network 110. Each of the professional computing device 102 and the patient computing device 104 may be embodied as a desktop, laptop, smartphone, or tablet computer that incorporates Wi-Fi functionality and/or cellular telephone functionality. Notably, the communication network 110 may use one or more of various communication types such as, for example and without limitation, wired, cellular and Wi-Fi communications. Moreover, each of the professional computing device 102 and the patient computing device 104 may be coupled to a power supply, which may be effectuated by way of a power cord, battery, and/or other means of supplying electrical power as may be available or otherwise desired.

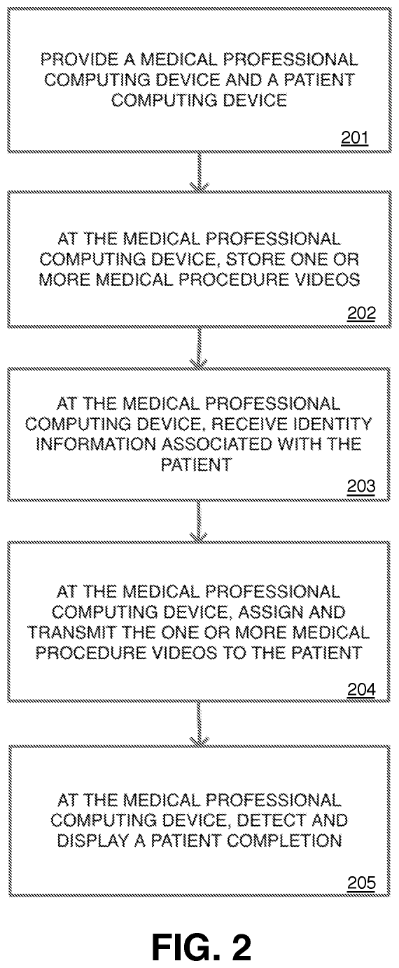

The medical professional computing device 102 and the patient computing device 104 may become members of a network that enables each to interact with one another and exchange information, such as identity information associated with the patient and one or more medical procedure videos, which may comprise one or more items. In this exemplary embodiment, the network may be facilitated by a website that is hosted by a network server. As such, the server may facilitate interaction among a limited group of members, as may be established by the medical professional itself. For purposes of the example presented in FIG. 1, the limited group of members may include the medical professional computing device 102 and the patient computing device 104. Additionally, the server may implement the system for enhanced retention of procedure-related information and facilitate sharing information relating to the one or more medical procedures. Specifically, the server may implement the steps outlined in FIG. 2. Accordingly, members of the network 110 may access the server using their devices 102 and 104 to obtain information, input information, receive notifications, such as text messages and push notifications regarding a patient completion of the one or more medical procedure videos.

In some embodiments, the medical professional computing device 102 may be operative to store the one or more medical procedure videos. The one or more medical procedure videos may be generally categorized as preoperative videos, operative videos, and postoperative videos. The preoperative videos may comprise home preparation videos, which may include information regarding home preparation prior to the one or more medical procedures so as to render the patient's home accessible after the one or more medical procedures. The preoperative videos may also include information regarding diet, permissible medications, as well as, hospital expectations. The operative videos may explain the one or more medical procedures recommended or prescribed to the patient, including how the one or more medical procedures are performed and any risks associated therewith. In addition, the postoperative videos may explain expectations following the one or more medical procedures.

The one or more medical procedure videos may comprise visual data and audio data. The visual data may comprise at least one video screen and the at least one video screen may have fifteen frames. The visual data may feature one or more of image content, animation content, and text content. The image content and/or the animation content may comprise at least one character, which may resemble a human or an animal. In certain embodiments, the at least one character may have a face. Moreover, the image content and/or the animation content may comprise medical equipment, tools, instruments, or other items, including those related to the one or more medical procedures. The animation content may display at between five to fifteen frames per second. In some exemplary embodiments, the animation content may display at eight frames per second.

The text content may comprise one or more words. In some embodiments, the one or more words may be associated with the one or more medical procedures. In this way, the text content may comprise key or highlight words associated with the one or more medical procedures. In further embodiments, the text content may coincide with the audio data and therefore, may appear on the at least one video screen while the audio data may simultaneously sound. In such embodiments, the text content may comprise a full sentence corresponding to the audio data. In other embodiments, the text content may comprise key or highlight words immediately following the audio data.

In embodiments where the at least one video screen may comprise three video screens, the text content may comprise up to 24 words across the three video screens. In some of such embodiments, the up to 24 words may comprise the full sentence. Further, the one or more medical procedure videos may be between one and five minutes in duration. In certain exemplary embodiments, the one or more medical procedure videos may be approximately two minutes in duration.

According to certain embodiments, the audio data may comprise speech content and non-speech content. The speech content may comprise at least one speaker. In some embodiments, the at least one speaker may comprise a female voice. In other embodiments, the at least one speaker may comprise a male voice or a gender-neutral voice. In further embodiments wherein the at least one speaker may comprise a female voice, the female voice may comprise a British female voice. Alternatively, the at least one speaker may comprise an American voice. In even further embodiments, the at least one speaker may comprise other types of voices as well based on a language and a dialect of the patient.

The non-speech content may comprise instrumental or computer music. In some exemplary embodiments, the instrumental music or the computer music may be non-dynamic. In these embodiments, the non-speech content may comprise minimal or no variation in volume between notes or phrases and therefore, the speech content may be easily heard over the non-speech content. Indeed, in such embodiments, the non-speech content may not be readily observed by the patient. Nonetheless, the non-speech content may help pace the one or more medical procedure videos and move the animation and/or the image content along.

The professional computing device 102 may be further operative to receive identity information associated with the patient. In some embodiments, the identity information may comprise the one or more medical procedures recommended or prescribed to the patient by the medical professional. Additionally, the identity information may comprise personal information about the patient. For instance, the identity information may comprise name, social security number or other identifying number, medical history, symptoms, clinical data, diagnoses, medications, treatment plants, laboratory and test results.

Also, the medical professional computing device 102 may be operative to assign and transmit the one or more medical procedure videos to the patient. In this manner, the medical professional computing device 102 may assign and transmit the one or more medical procedure videos based on the one or more medical procedures recommended or prescribed to the patient. The medical professional computing device 102 may then be operative to detect and display a patient completion. The patient completion may comprise the patient viewing the one or more medical procedure videos. In some embodiments, the patient completion may comprise the patient viewing all of the one or more medical procedure videos. In other embodiments, the patient completion may comprise the patient viewing one or more of the one or more medical procedure videos. In addition, the patient completion may be defined by the medical professional.

The patient computing device 104 may be operative to receive and transmit the identity information associated with the patient. As such, the patient computing device 104 may receive identifying information, such as name or social security number, from the patient. In turn, the patient computing device 104 may be operative to transmit the identity information to the medical professional computing device.

The patient computing device 104 may be further operative to receive and display the one or more medical procedure videos. In this manner, the patient computing device 104 may be operative to receive the one or more medical procedure videos from the professional computing device 102. Again, the patient computing device 104 may be operative to display the one or more medical procedure videos based on the one or more medical procedures recommended or prescribed by the medical professional to the patient.

In accordance with certain embodiments, the patient computing device 104 may be operative to notify and transmit the patient completion. In particular, responsive to the patient viewing one or more of the one or more medical procedure videos, the patient computing device 104 may be operative to notify the patient and/or the medical professional of the patient completion. Along these lines, the patient computing device 104 may be operative to notify the patient of a partial completion of the one or more medical procedure videos and thereby, may remind the patient to view the one or more medical procedure videos. Similarly, the patient computing device 104 may be operative to transmit the patient completion to the medical professional and/or the medical professional computing device 102.

FIGS. 2-4 are flowcharts depicting an exemplary embodiment of the system and method for enhanced retention of procedure-related information, such as may be performed by the medical professional computing device 102 and the patient computing device 104 of FIG. 1. As shown in FIG. 2, the method may include the steps of: providing a computing device associated with a medical professional and a computing device associated with a patient (block 201); storing, at the computing device associated with the medical professional, one or more medical procedure videos (block 202); receiving, at the computing device associated with the medical professional, identity information associated with the patient (block 203); assigning and transmitting, at the computing device associated with the medical professional, the one or more medical procedure videos to the patient (block 204); and detecting and displaying, at the computing device associated with the medical professional, a patient completion, wherein the patient completion comprises the patient viewing the one or more medical procedure videos (block 205).

Storing the one or more medical procedure videos (block 202) may comprise storing only the one or more medical procedure videos relevant to the patient. In other embodiments, storing the one or more medical procedure videos (block 202) may comprise storing all of the one or more medical procedure videos, including those relevant to the patient, but only assigning and transmitting the one or more medical procedure videos (block 204) recommended or prescribed by the medical professional to the patient.

Receiving the identity information (block 203) may comprise receiving the one or more medical procedures recommended or prescribed by the medical professional to the patient. Receiving the identity information (block 203) may also comprise receiving personal and/or identifying information about the patient. For example, the identity information may comprise name, medical history, diagnoses, symptoms, laboratory and test results, medical professional notes, and treatment plans.

Assigning and transmitting the one or more medical procedure videos to the patient (block 204) may further comprise determining which of the one or more medical procedure videos to assign to the patient. Determining which of the one or more medical procedure videos to assign to the patient may be defined by the medical professional. In some embodiments, determining which of the one or more medical procedure videos to assign to the patient may depend on the one or more medical procedure videos prescribed or recommended by the medical professional. Further, determining which of the one or more medical procedure videos to assign to the patient may depend on other identity information, such as the diagnoses or the treatment plan.

Detecting the patient completion (block 205) may comprise the patient viewing the one or more medical procedure videos. According to certain embodiments, detecting the patient completion (block 205) may comprise the patient viewing all of the one or more medical procedure videos. In other embodiments, detecting the patient completion (block 205) may comprise the patient viewing a majority of the one or more medical procedure videos. In alternate embodiments, detecting the patient completion (block 205) may comprise the patient viewing one or more of the one or more medical procedure videos. Furthermore, detecting the patient completion (block 205) may be defined by the medical professional.

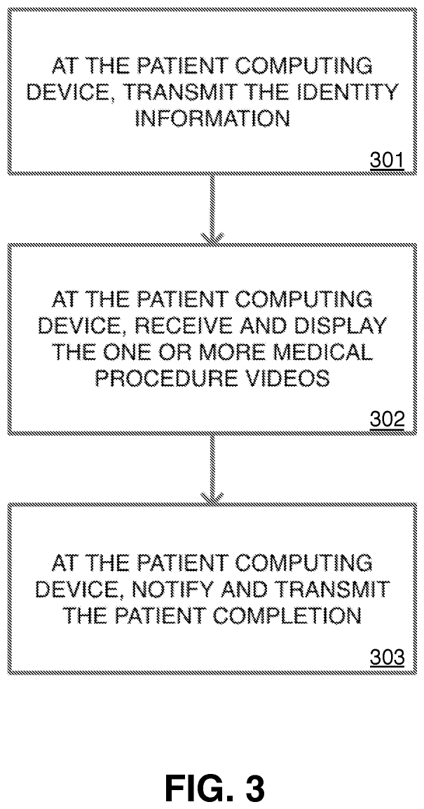

As shown in FIG. 3, the method may further include the steps of: transmitting, at the computing device associated with the patient, the identity information associated with the patient (block 301); receiving and displaying, at the computing device associated with the patient, the one or more medical procedure videos (block 302); and notifying and transmitting, at the computing device associated with the patient, the patient completion (block 303).

Transmitting the identity information (block 301) may comprise receiving an invitation. In such embodiments, the invitation may comprise a link or request to access the one or more medical procedure videos. Transmitting the identity information (block 301) may further comprise inputting of a passcode, a password, or other information by the patient.

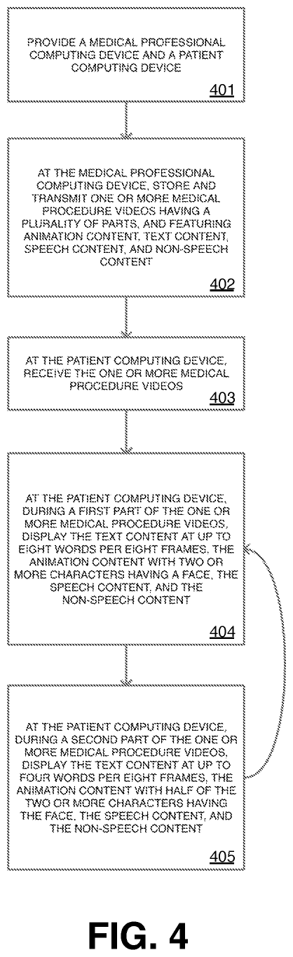

As shown in FIG. 4, the method may include the steps of: providing a computing device associated with a medical professional and a computing device associated with a patient (block 401); storing and transmitting, at the computing device associated with the medical professional, one or more medical procedure videos having a plurality of parts, and featuring animation content, text content, speech content, and non-speech content (block 402); receiving, at the computing device associated with the patient, the one or more medical procedure videos (block 403); during a first part of the one or more medical procedure videos, displaying, at the computing device associated with the patient, the text content at up to eight words per eight frames, the animation content with two or more characters having a face, the speech content, and the non-speech content (block 404); during a second part of the one or more medical procedure videos, displaying, at the computing device associated with the patient, the text content at up to four words per eight frames, the animation content with half of the two or more characters having the face, the speech content, and the non-speech content (block 405).

Storing and transmitting, at the computing device associated with the medical professional, the one or more medical procedure videos having the plurality of parts, and featuring animation content, text content, speech content, and non-speech content (block 402) may further comprise the one or more medical procedure videos having the first part and the second part. In additional embodiments, the plurality of parts may also comprise a third part and a fourth part. In still further embodiments, the plurality of parts may comprise additional parts, such as a fifth part and a sixth part, as well. In such embodiments comprising the third part, the fourth part, and even the fifth part and the sixth part, the steps provided in blocks 404-405 may be repeated sequentially. In certain embodiments, the one or more medical procedure videos may be two minutes in duration. Furthermore, in some exemplary embodiments, the text content and the speech content may match.

The animation content may comprise the two or more characters having the face. A person of ordinary skill in the art will recognize the face may comprise a human face or an animal face and therefore, may comprise two eyes, a nose, and a mouth. In some embodiments, such as during the first part, displaying the animation content (block 404) may comprise all of the two or more characters having the face. In addition, during the first part, displaying the text content (block 404) may comprise displaying the text content at up to eight words per eight frames. In other embodiments, such as during the second part, displaying the animation content (block 405) may comprise only half of the two or more characters having the face. Moreover, during the second part, displaying the text content (block 405) may comprise displaying the text content at four words per eight frames. In these embodiments, displaying the animation content and the text content in the aforementioned manner (blocks 404-405) may cause retention of procedure-related information to be 580% greater than displaying the text content alone and may be 30% more effective than displaying the animation content alone, as may be commonly known in the art.

If embodied in software, it should be noted that each block depicted in the accompanying flowcharts of FIGS. 2-4 represents a module, segment, or portion of code that comprises program instructions stored on a non-transitory computer readable medium to implement the specified logical functions. In this regard, the program instructions may be embodied in the form of source code that comprises statements written in a programming language or machine code that comprises numerical instructions recognizable by a suitable execution system such as the computing devices 102 and 104. The machine code may be converted from the source code, etc. If embodied in hardware, each block may represent a circuit or a number of interconnected circuits to implement the specified logical functions. Additionally, although the flowcharts of FIGS. 2-4 show specific orders of execution, it is to be understood that the orders of execution may differ.

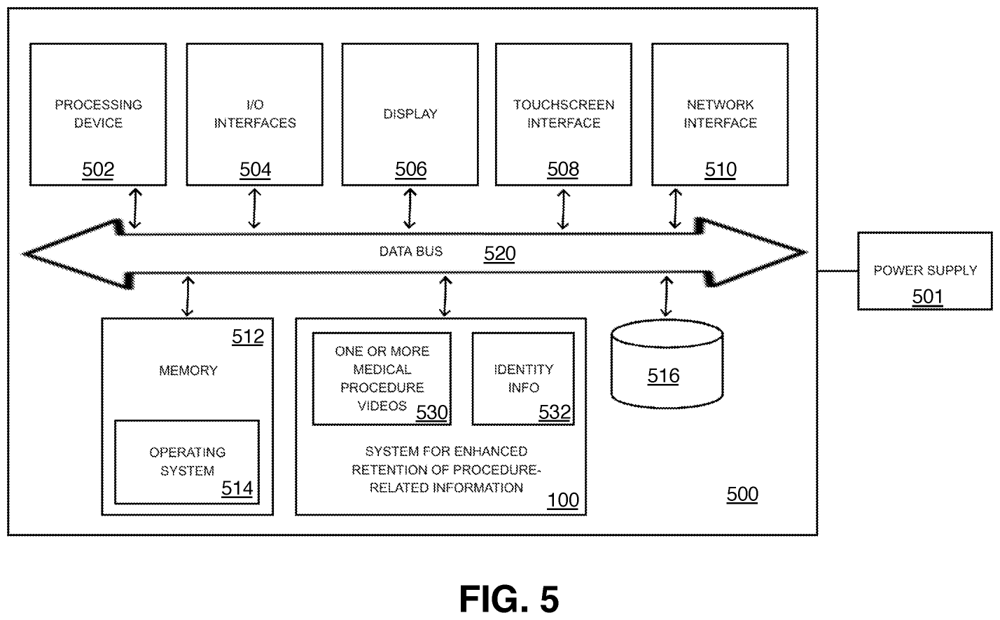

FIG. 5 illustrates an exemplary medical professional computing device 500 configured to implement the system for enhanced retention of procedure-related information. As described earlier, medical professional computing device 500 may be a desktop computer, laptop computer, tablet computer or smartphone but may also be embodied in any one of a wide variety of wired and/or wireless computing devices known to those skilled in the art. As shown in FIG. 5, medical professional computing device 500 may include a processing device (processor) 502, input/output interfaces 504, a display 506, a touchscreen interface 508, a network interface 510, a memory 512, and operating system 514, and a mass storage 516, with each communicating across a local data bus 520. Additionally, medical professional computing device 500 may incorporate the system for enhanced retention of procedure-related information 100, which is depicted as including one or more medical procedure videos 530 and identity information 532, although the location of information 530 and 532 may vary. The medical professional computing device 500 may further comprise a power supply 501.

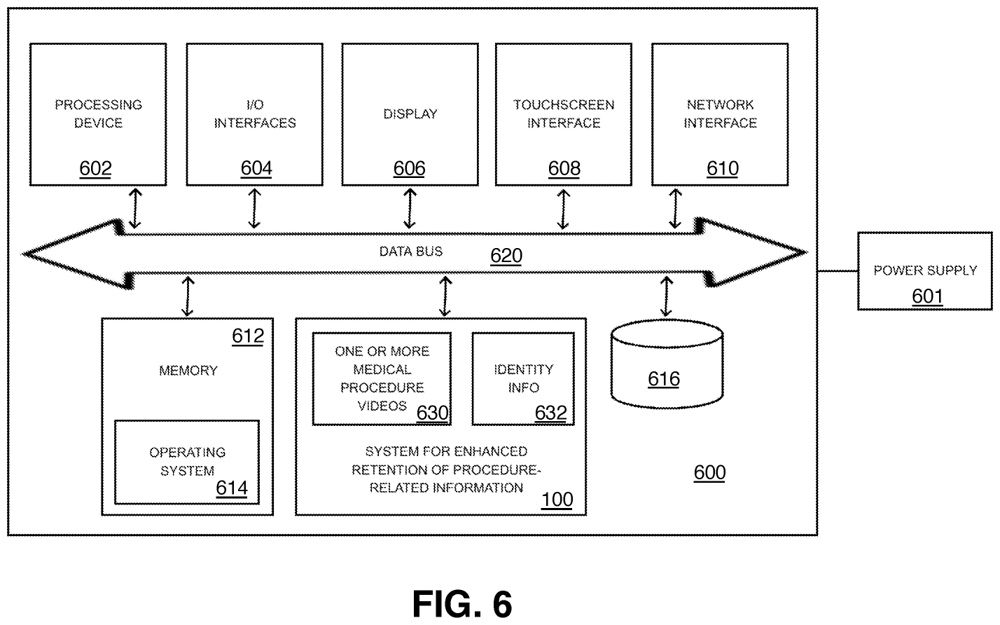

FIG. 6 illustrates an exemplary patient computing device 600 configured to implement the system for enhanced retention of procedure-related information. As described earlier, patient computing device 600 may be a desktop computer, laptop computer, tablet computer or smartphone but may also be embodied in any one of a wide variety of wired and/or wireless computing devices known to those skilled in the art. As shown in FIG. 6, patient computing device 600 may include a processing device (processor) 602, input/output interfaces 604, a display 606, a touchscreen interface 608, a network interface 610, a memory 612, and operating system 614, and a mass storage 616, with each communicating across a local data bus 620. Additionally, patient computing device 600 may incorporate the system for enhanced retention of procedure-related information 100, which is depicted as including one or more medical procedure videos 630 and identity information 632, although the location of information 630 and 632 may vary. The patient computing device 600 may further comprise a power supply 601.

The processing devices 502, 602 may include any custom made or commercially available processor, a central processing unit (CPU) or an auxiliary processor among several processors associated with the medical professional computing device 500 and the patient computing device 600, a semiconductor based microprocessor (in the form of a microchip), a macroprocessor, one or more application specific integrated circuits (ASICs), a plurality of suitably configured digital logic gates, and other electrical configurations comprising discrete elements both individually and in various combinations to coordinate the overall operation of the system.

The memory 512, 612 may include any one of a combination of volatile memory elements (e.g., random-access memory (RAM, such as DRAM, and SRAM, etc.)) and nonvolatile memory elements. The memory may typically comprise native operating system 514, 614 one or more native applications, emulation systems, or emulated applications for any of a variety of operating systems and/or emulated hardware platforms, emulated operating systems, etc. For example, the applications may include application specific software which may comprise some or all the components of the medical professional computing device 500 and the patient computing device 600. In accordance with such embodiments, the components are stored in memory and executed by the processing device. Note that although depicted separately in FIGS. 5-6, the system and method for enhanced retention of procedure-related information 100 may be resident in memory such as memory 512, 612. Touchscreen interfaces 508, 608 may be configured to detect contact within the display area of the displays 506, 606 and may provide such functionality as on-screen buttons, menus, keyboards, etc. that allows users to navigate user interfaces by touch.

One of ordinary skill in the art will appreciate that the memory 512, 612 may, and typically will, comprise other components which have been omitted for purposes of brevity. Note that in the context of this disclosure, a non-transitory computer-readable medium stores one or more programs for use by or in connection with an instruction execution system, apparatus, or device. With further reference to FIGS. 5-6, network interface devices 510, 610 may comprise various components used to transmit and/or receive data over a networked environment such as depicted in FIG. 1. When such components are embodied as an application, the one or more components may be stored on a non-transitory computer-readable medium and executed by the processing device.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. For example, the one or more medical procedure videos may be substituted for other types of videos comprising other types of information as well, including non-medical information. Accordingly, it is intended that the invention not be limited, except as by the appended claims.

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the system and method for enhanced retention of procedure-related information with which that terminology is associated. In general, the terms used in the following claims should not be constructed to limit the system and method for enhanced retention of procedure-related information to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the system and method for enhanced retention of procedure-related information is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the system and method for enhanced retention of procedure-related information.

What is claimed is:

1. A system for enhanced retention of procedure-related information, comprising
a computing device associated with a medical professional, operative to
store one or more medical procedure videos, the one or more medical procedure videos comprising visual data and audio data;
receive identity information associated with a patient;
assign and transmit the one or more medical procedure videos to the patient;
detect and display a patient completion, wherein the patient completion comprises the patient viewing the one or more medical procedure videos;
a computing device associated with the patient, operative to
receive and transmit the identity information associated with the patient;
receive and display the one or more medical procedure videos; and
notify and transmit the patient completion.

2. The system of claim 1, wherein the visual data comprises at least one video screen having 15 frames and featuring one or more of image content, animation content, and text content.

3. The system of claim 2, wherein the animation content displays at between 5 to 15 frames per second.

4. The system of claim 2, wherein the animation content displays at 8 frames per second.

5. The system of claim 2, wherein one or more of the image content and the animation content comprises at least one character having a face.

6. The system of claim 2, wherein the at least one video screen comprises three video screens and the text content comprises up to 24 words across the three video screens.

7. The system of claim 1, wherein the audio data comprises speech content and non-speech content.

8. The system of claim 7, wherein the speech content comprises at least one speaker.

9. The system of claim 8, wherein the at least one speaker has a female voice.

10. The system of claim 9, wherein the female voice is a British female voice.

11. The system of claim 7, wherein the non-speech content is non-dynamic and comprises instrumental music or computer music.

12. A method for enhanced retention of preoperative and postoperative information, comprising
providing a computing device associated with a medical professional and a computing device associated with a patient;
at the computing device associated with the medical professional,
storing one or more medical procedure videos;
receiving identity information associated with the patient;
assigning and transmitting the one or more medical procedure videos to the patient;
detecting and displaying a patient completion, wherein the patient completion
comprises the patient viewing the one or more medical procedure videos;
at the computing device associated with the patient;
receiving and transmitting the identity information associated with the patient;
receiving and displaying the one or more medical procedure videos; and
notifying and transmitting the patient completion.

13. The method of claim 12, wherein the one or more medical procedure videos comprise visual data having at least one video screen with 15 frames and audio data.

14. The method of claim 13, wherein the visual data comprises one or more of image content, animation content, and text content, and wherein the animation content displays at 8 frames per second.

15. The method of claim 13, wherein the at least one video screen comprises three video screens and the text content comprises up to 24 words across the three video screens.

16. A method for enhanced retention of procedure-related information, comprising
providing a computing device associated with a medical professional and a computing device associated with a patient;
at the computing device associated with the medical professional, storing and transmitting one or more medical procedure videos having a plurality of parts, and featuring animation content, text content, speech content, and non-speech content;
at the computing device associated with the patient,
a) receiving the one or more medical procedure videos;
b) during a first part of the one or more medical procedure videos, displaying the text content at up to eight words per eight frames, the animation content with two or more characters having a face, the speech content, and the non-speech content; and
c) during a second part of the one or more medical procedure videos, displaying the text content at up to four words per eight frames, the animation content with half of the two or more characters having the face, the speech content, and the non-speech content.

17. The method of claim 16, wherein each of the first part and the second part are 30 seconds in duration.

18. The method of claim 17, wherein the one or more medical procedure videos are two minutes in duration and further comprise a third part and a fourth part.

19. The method of claim 16, wherein during the first part, all of the one or more characters have the face.

20. The method of claim 16, wherein the text content and the speech content match.
